(19) United States
(12) Patent Application Publication
(10) Pub. No.: US 2013/0158355 A1
(43) Pub. Date: Jun. 20, 2013

(54) TWO-WAY ENDOSCOPE STEERING MECHANISM AND FOUR-WAY ENDOSCOPE STEERING MECHANISM

(71) Applicant: WEI-TENG LIN, NEW TAIPEI CITY (TW)

(72) Inventor: WEI-TENG LIN, NEW TAIPEI CITY (TW)

(73) Assignee: PIONEER MEDICAL INSTRUMENT CO., LTD., NEW TAIPEI CITY (TW)

(21) Appl. No.: 13/328,005

(22) Filed: Dec. 16, 2011

(51) Int. Cl. A61B 1/008

(52) U.S. Cl. 600/141

(57) ABSTRACT

The present invention discloses a two-way endoscope steering mechanism and four-way endoscope steering mechanism, and the two-way endoscope steering mechanism includes a hollow steering member, a two-way flexible plastic member and a guiding wire member. The hollow steering member has a hollow positioning portion and a connecting portion. The guiding wire member is passed and installed in the hollow positioning portion. The densely wound coil is coupled to an end of a steering module, and the steering module is formed by arranging and coupling the two-way flexible plastic member to the hollow steering member, and the densely wound coil is sheathed on the guiding wire member, such that the guiding wire member can be pulled to drive the steering module to turn in a direction.

### BACKGROUND OF THE INVENTION

1. Field of the Invention

The present invention relates to an endoscopic device, in particular to the field of endoscope steering mechanisms.

2. Description of the Related Art

In general, a conventional endoscope steering mechanism includes a plurality of hollow metal structures, and each metal structure has a rivet hole corresponding to a rivet for steering the metal structure. Therefore, a high precision is required for the manufacture, and the high-precision manufacture usually incurs a high production cost and a complicated manufacturing procedure. In view of the drawbacks of the conventional endoscope steering mechanism, the inventor of the present invention developed an endoscope steering mechanism in accordance with the present invention to overcome the aforementioned drawbacks.

### SUMMARY OF THE INVENTION

Therefore, it is a primary objective of the present invention to provide a two-way endoscope steering mechanism and a four-way endoscope steering mechanism capable of turning a steering module in a first direction or a second direction, or turning the steering module in a first, second, third or fourth direction by a simple structure.

To achieve the foregoing objective, the present invention provides a two-way endoscope steering mechanism, comprising a plurality of hollow steering members, a plurality of two-way flexible plastic members and a plurality of guiding wire members. Each hollow steering member has a hollow positioning portion and a connecting portion. The connecting portion includes a first connecting portion, a second connecting portion, a third connecting portion and a fourth connecting portion, wherein the first connecting portion and the second connecting portion are arranged opposite to each other and disposed on a side of the hollow steering member; the third connecting portion and the fourth connecting portion are arranged opposite to each other and disposed on the other side of the hollow steering member; and the first connecting portion and the second connecting portion are arranged opposite to the third connecting portion and the fourth connecting portion respectively. The two-way flexible plastic member includes a first two-way flexible plastic member and a second two-way flexible plastic member, and each two-way flexible plastic member can be partially bent, and the bending and steering of the plurality of two-way flexible plastic members are accumulated to define a partial bending of each two-way flexible plastic member. An end of the first two-way flexible plastic member is coupled to the first connecting portion, and the other end of the first two-way flexible plastic member is coupled to the third connecting portion adjacent to another hollow steering member. An end of the second two-way flexible plastic member is coupled to the second connecting portion, and the other end of the second two-way flexible plastic member is coupled to the fourth connecting portion adjacent to another hollow steering member. The guiding wire member is passed and installed into the corresponding hollow positioning portion. The densely wound coil is coupled to an end of a steering module, and the steering module is formed by arranging and coupling the two-way flexible plastic members to the hollow steering members, and the densely wound coil is extended in an opposite direction from an end of the steering module and sheathed on the guiding wire member. If one of the guiding wire members is pulled, the first two-way flexible plastic member and the second two-way flexible plastic member are pulled to bend towards the first direction or second direction to turn the steering module.

To achieve the foregoing objective, the present invention further provides a four-way endoscope steering mechanism, wherein one of the guiding wire members can be pulled to drive the guiding wire member to align with the corresponding hollow steering member, so that the two-way flexible plastic member on a side of the hollow steering member can be bent in a first direction or a second direction, or the two-way flexible plastic members on the other side of the hollow steering member can be bent in a third direction or a fourth direction to turn the steering module.

In summation, the two-way endoscope steering mechanism and the four-way endoscope steering mechanism of the present invention have the following advantage:
(1) A portion of the two-way flexible plastic member can be bent, and the plurality of hollow steering members and the plurality of two-way flexible plastic members can be used for turning the steering module. The invention provides an endoscope steering mechanism with a simple structure and a low price, and capable of simplifying the manufacturing procedure without hindering the bending of an endoscope.
(2) The densely wound coils sheathed onto the corresponding guiding wire members can prevent worn-outs when the guiding wire member is in contact with the hollow pipe, and the reaction force produced by applying a force to the guiding wire member is exerted onto the densely wound coil to turn the steering module.
(3) The hollow positioning portion is an open slot having an opening that defines a rotation angle with respect to an opening of an adjacent open slot, so that the guiding wire member can be latched into the open slot to achieve a positioning effect as well as an easy repair and maintenance by visual inspections.

### BRIEF DESCRIPTION OF THE DRAWINGS

FIG. 1 is a perspective view of a two-way endoscope steering mechanism applied in an endoscope in accordance with the present invention;

FIG. 2 is a schematic view of operating a two-way endoscope steering mechanism in accordance with a first preferred embodiment of the present invention;

FIG. 3 is a perspective view of a four-way endoscope steering mechanism in accordance with a second preferred embodiment of the present invention;

FIG. 4 is a schematic view of operating a four-way endoscope steering mechanism to achieve a viewing angle in accordance with the second preferred embodiment of the present invention;

FIG. 5 is a schematic view of operating a four-way endoscope steering mechanism to achieve another viewing angle in accordance with the second preferred embodiment of the present invention;

FIG. 6 is a perspective view of a two-way endoscope steering mechanism in accordance with a third preferred embodiment of the present invention; and

FIG. 7 is a perspective view of a four-way endoscope steering mechanism in accordance with a fourth preferred embodiment of the present invention.

### DETAILED DESCRIPTION OF THE PREFERRED EMBODIMENTS

The technical content of the present invention will become apparent by the detailed description of the following embodiments and the illustration of related drawings as follows.

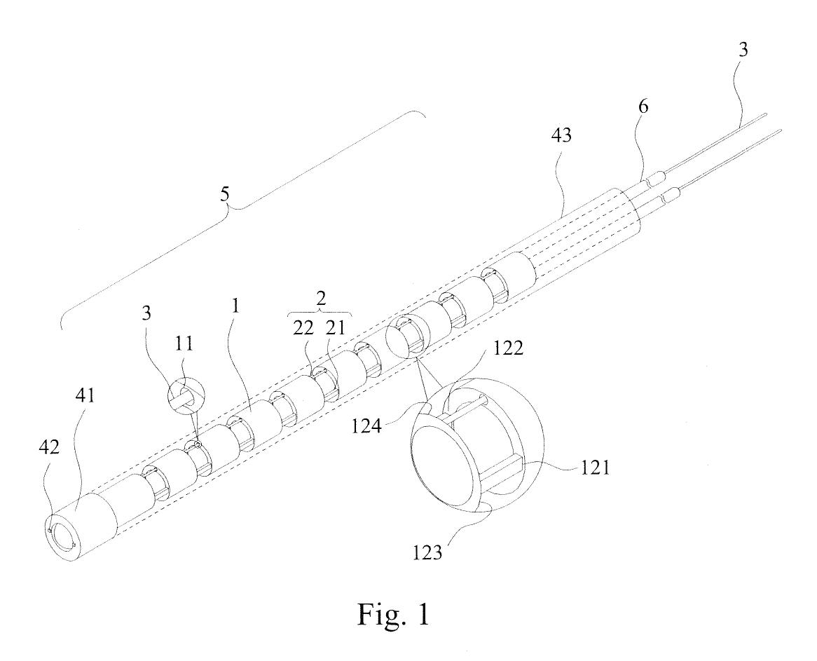

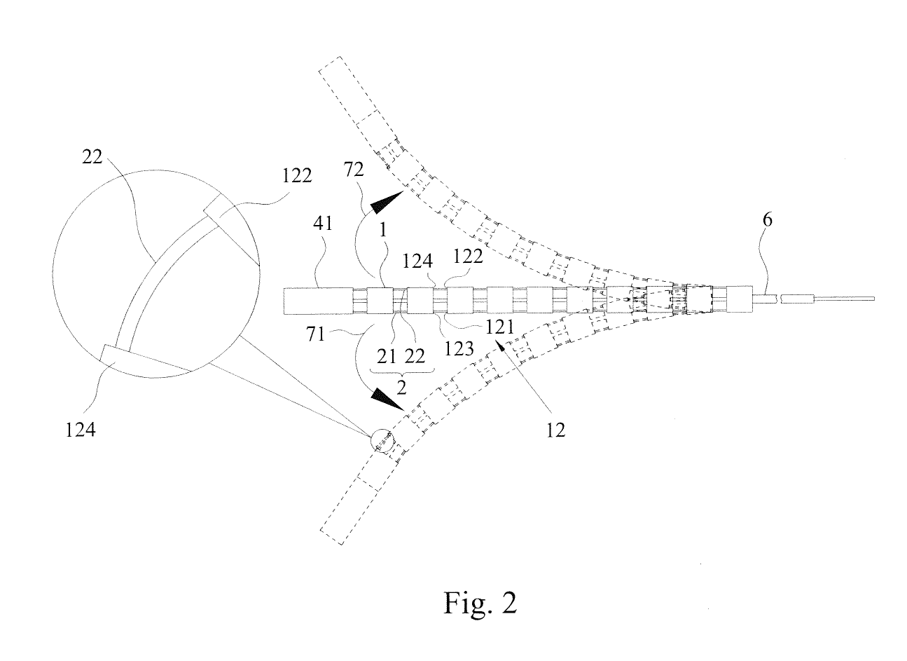

With reference to FIGS. 1 and 2 for a perspective view of a two-way endoscope steering mechanism applied in an endoscope in accordance with the present invention and a schematic view of operating a two-way endoscope steering mechanism in accordance with a first preferred embodiment of the present invention respectively, the two-way endoscope steering mechanism comprises a plurality of hollow steering members 1, a plurality of two-way flexible plastic members 2 and a plurality of guiding wire members 3.

The hollow steering member 1 includes a plurality of hollow positioning portions 11 and a plurality of connecting portions 12, and each hollow positioning portion 11 is a through hole or an opening. In this preferred embodiment, the hollow positioning portions 11 are disposed in the hollow steering member 1, and the connecting portions 12 are disposed on a side of the hollow steering member 1, and the hollow steering member 1 is made of metal, plastic, or memory alloy.

The connecting portion 12 includes a first connecting portion 121, a second connecting portion 122, a third connecting portion 123 and a fourth connecting portion 124, wherein the first connecting portion 121 and the second connecting portion 122 are arranged opposite to each other and disposed on a side of the hollow steering member 1, and the third connecting portion 123 and the fourth connecting portion 124 are arranged opposite to each other and disposed on the other side of the hollow steering member 1. The first connecting portion 121 and the second connecting portion 122 are arranged opposite to the third connecting portion 123 and the fourth connecting portion 124 respectively.

Each two-way flexible plastic member 2 includes a first two-way flexible plastic member 21 and a second two-way flexible plastic member 22, and the two-way flexible plastic member 2 can be partially bent, and the bending and steering of the plurality of two-way flexible plastic members 2 can be accumulated to define the partial bending of the two-way flexible plastic member 2. An end of the first two-way flexible plastic member 21 is coupled to the first connecting portion 121, and the other end of the first two-way flexible plastic member 21 is coupled to adjacent to the third connecting portion 123 of another hollow steering member 1. An end of the second two-way flexible plastic member 22 is coupled to the second connecting portion 122, and the other end of the second two-way flexible plastic member 22 is coupled to the fourth connecting portion 124 adjacent to another hollow steering member 1.

In this preferred embodiment, an end of the first two-way flexible plastic member 21 is integrally formed with the first connecting portion 121 and the third connecting portion 123, and an end of the second two-way flexible plastic member 22 is integrally formed with the second connecting portion 122 and the fourth connecting portion 124. In other words, the connection can be achieved by an integral formation or an adhesive connection. In addition, the two-way flexible plastic member 2 can be made of plastic and has a shape of a plate, a pillar, or a sheet.

The guiding wire member 3 is passed and installed into the plurality of corresponding hollow positioning portions 11. The endoscope steering mechanism further comprises a camera device 41 and a light emitting element 42 installed at a front end of the endoscope steering mechanism. The endoscope steering mechanism can be wrapped by a hollow tube 43, and the hollow tube 43 is extended axially towards an end of the endoscope steering mechanism. With the installation of the camera device 41, the light emitting element 42, and the hollow tube 43, an endoscopic device is produced. Wherein, an end of each guiding wire member 3 is fixed to the hollow steering member 1, the camera device 41 or a casing covering the camera device 41 (as shown in FIG. 1).

The densely wound coil 6 is coupled to an end of a steering module 5, and the steering module 5 is arranged and coupled to the plurality of hollow steering members 1 through the plurality of two-way flexible plastic members 2, and the densely wound coil 6 is extended from an end of steering module 5 in an opposite direction of the steering module 5 and sheathed on the corresponding guiding wire member 3 to prevent any worn-out produced when the guiding wire member 3 is in contact with the hollow tube 43 and turn the steering module 5. During use, a user holds the bottom of the densely wound coil 6 by a left hand, and pulls the guiding wire member 3 by a right hand, and the reaction force produced by pulling the guiding wire member 3 by the right hand and the grip of the bottom of the densely wound coil 6 by the left hand can abut against the top of the densely wound coil 6 at an end of the steering module 5 to produce a reaction force to turn the steering module 5.

Wherein, if one of the guiding wire members 3 is pulled, the first two-way flexible plastic member 21 and second two-way flexible plastic member 22 are driven to bend in a first direction 71 or a second direction 72 to turn the steering module 5 in the first direction 71 or second direction 72.

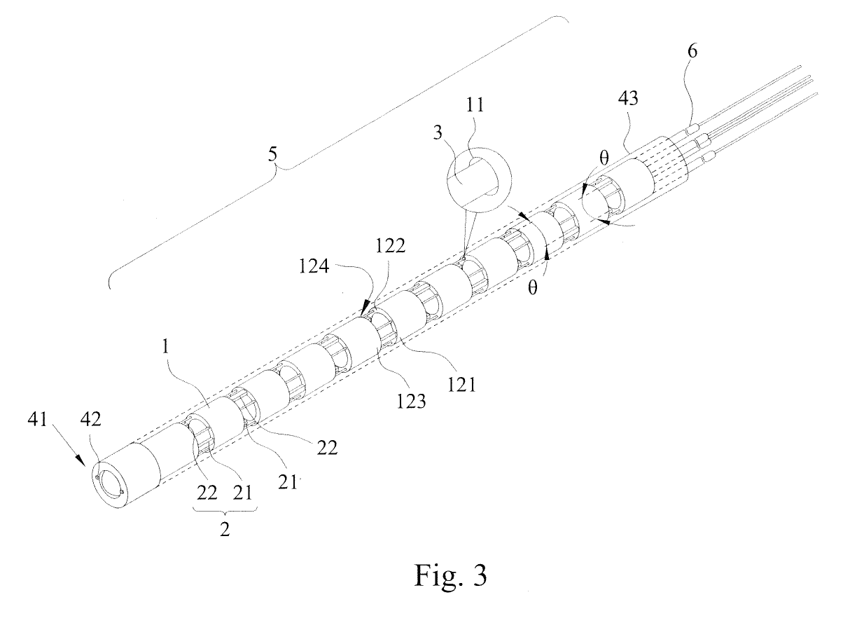

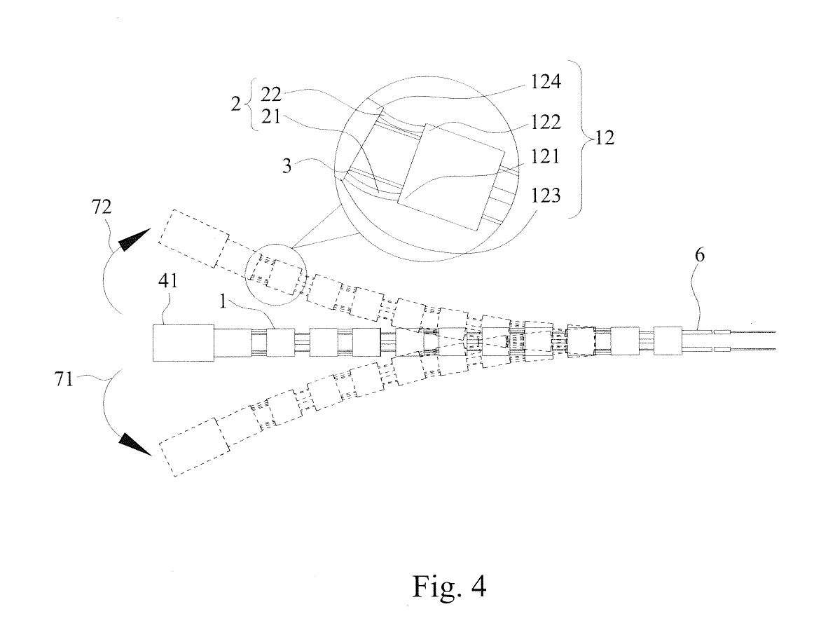

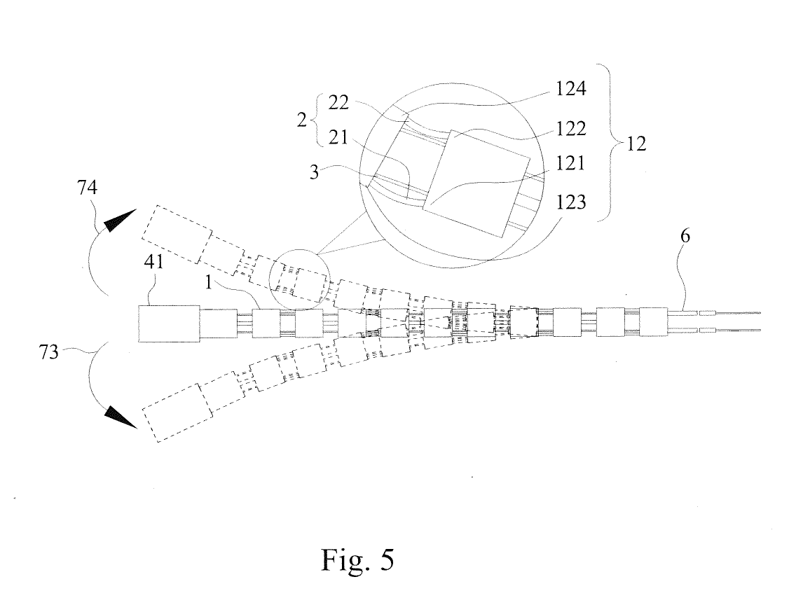

With reference to FIGS. 3 to 5 for a perspective view of a four-way endoscope steering mechanism in accordance with the second preferred embodiment of the present invention and schematic views of operating a four-way endoscope steering mechanism to achieve another viewing angle in accordance with the second preferred embodiment of the present invention the present invention respectively, the four-way endoscope steering mechanism comprises a plurality of hollow steering members 1, a plurality of two-way flexible plastic members 2 and a plurality of guiding wire members 3.

The hollow steering members 1 are the same as those of the first preferred embodiment, and thus will not be described again.

The connecting portion 12 includes a first connecting portion 121, a second connecting portion 122, a third connecting portion 123 and a fourth connecting portion 124, wherein the first connecting portion 121 and the second connecting portion 122 are arranged opposite to each other and disposed on a side of the hollow steering member 1, and the third connecting portion 123 and the fourth connecting portion 124 are arranged opposite to each other and disposed on the other side of the hollow steering member 1. In this preferred embodiment, the third connecting portion 123 and the first connecting portion 121 are arranged with an angle θ substantially equal to 90 degrees with respect to each other, and the fourth connecting portion 124 and the second connecting portion 122 are arranged with an angle θ substantially equal to 90 degrees with respect to each other. The two-way flexible plastic member 2 on a side of the hollow steering member 1 and the two-way flexible plastic member 2 on the other side of the hollow steering member 1 are arranged with an angle θ equal to 90 degrees with respect to each other.

One of the two-way flexible plastic members 2 has a first two-way flexible plastic member 21 and a second two-way flexible plastic member 22. The two-way flexible plastic member 2 can be partially bent, and the bending and turning of the plurality of two-way flexible plastic members 2 are accumulated to define a partial bending of the two-way flexible plastic member 2. An end of each first two-way flexible plastic member 21 is coupled to the first connecting portion 121, and the other end of each first two-way flexible plastic member 21 is coupled to the third connecting portion 123 adjacent to another hollow steering member 1. An end of each second two-way flexible plastic member 22 is coupled to the second connecting portion 122, and the other end of each second two-way flexible plastic member 22 is coupled to the fourth connecting portion 124 adjacent to another hollow steering member 1.

In this preferred embodiment, an end of the first two-way flexible plastic member 21 is integrally formed with the first connecting portion 121 and the third connecting portion 123, and an end of the second two-way flexible plastic member 22 is integrally formed with the second connecting portion 122 and the fourth connecting portion 124, and the two-way flexible plastic member 2 on a side of the hollow steering member 1 is perpendicular to the two-way flexible plastic member 2 on the other side of the hollow steering member L In addition, the type and material of the two-way flexible plastic member 2 are the same as those of the first preferred embodiment.

The way of passing, installing and fixing the guiding wire member 3 is the same as that of the first preferred embodiment. The components installed at the front end of the endoscope steering mechanism are the same as those of the first preferred embodiment to form the endoscopic device (as shown in FIG. 1), and these components thus will not be described again. The way of coupling the densely wound coil 6 is the same as that of the first preferred embodiment, and thus will not be described again.

Wherein, one of the guiding wire members 3 is pulled, so that the guiding wire member 3 drives the corresponding hollow steering member 1 to bend the two-way flexible plastic member 2 on a side of the hollow steering member 1 in a first direction or a second direction, or bend the two-way flexible plastic member 2 on the other side of the hollow steering member 1 in a third direction 73 or a fourth direction 74 to turn the steering module 5.

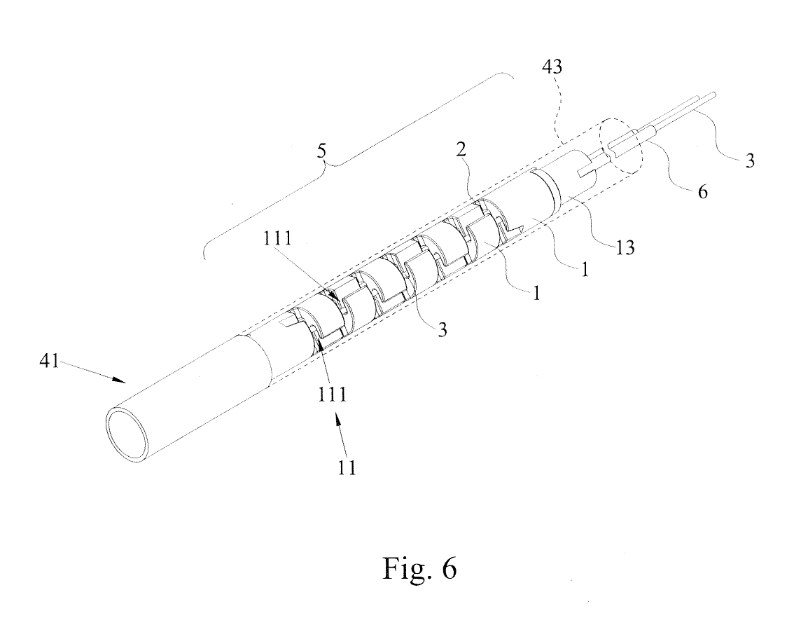

With reference to FIG. 6 for a perspective view of a two-way endoscope steering mechanism in accordance with the third preferred embodiment of the present invention together with FIGS. 1 and 2, the structure of this preferred embodiment is substantially the same as the structure illustrated in FIG. 1 except that each hollow positioning portion 11 of this preferred embodiment is an open slot 111, and an opening of the open slot 111 is formed with a rotating angle with an opening of an adjacent open slot 111, so that the guiding wire member 3 can be positioned in the hollow positioning portion 11. In this preferred embodiment, the opening of the open slot 111 and the opening of the adjacent open slot 111 are arranged with a rotation angle θ substantially equal to 180 degrees, and the guiding wire member 3 can be latched into the open slot 111 to achieve the positioning effect.

In addition, when the aforementioned open slot 111 of the two-way endoscope steering mechanism is installed, maintained or repaired, an operator can inspect the whole guiding wire member 3 by a visual inspection from different viewing angles to examine whether or not the guiding wire member 3 is damaged or failed to improve the convenience of inspecting the guiding wire member 3 and enhance the yield rate.

In addition, the steering module 5 further includes a support portion 13 formed at an end of the steering module 5, wherein the support portion 13 is another independent support member, and an end of the support portion 13 is coupled to the plurality of hollow steering members 1, and the bottom of the hollow steering member arranged at the distal end is coupled directly to the support portion 13 by soldering, tying, snapping, or latching). A reaction force is produced by pulling the guiding wire member 3 by a right hand, and the bottom of the densely wound coil 6 is grabbed by a left hand, so that the top of the densely wound coil 6 abuts against the end of the steering module 5 to produce a reaction force to turn the steering module 5.

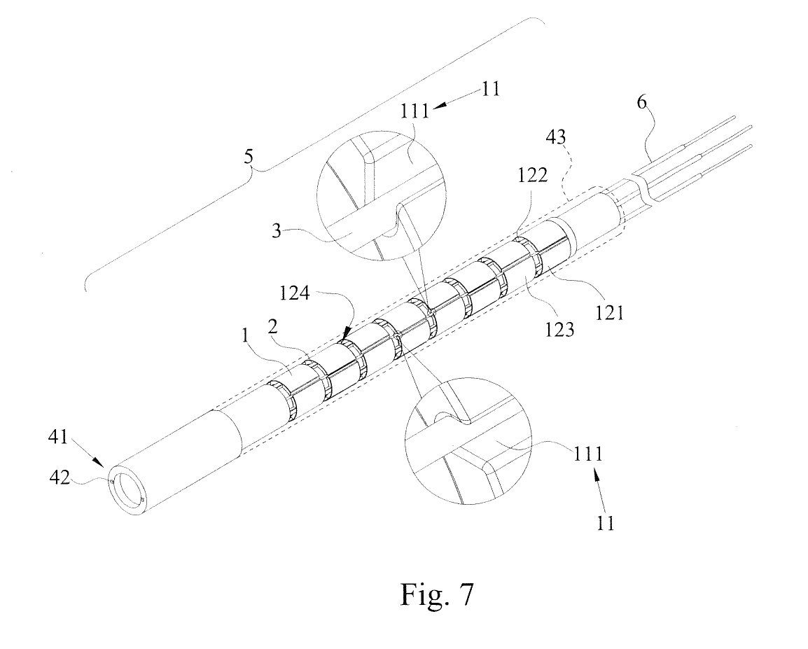

With reference to FIG. 7 for a perspective view of a four-way endoscope steering mechanism in accordance with the fourth preferred embodiment of the present invention, together with FIGS. 6, 3 and 4, the structure of this preferred embodiment is substantially the same as that illustrated in FIG. 3, except that each hollow positioning portion 11 of this preferred embodiment is an open slot 111, and the opening of each open slot 111 and the opening of the adjacent open slot 111 have a rotation angle θ substantially equal to 90 degrees, so that the guiding wire member 3 can be latched into the open slot 111 to achieve the positioning effect.

What is claimed is:

1. A two-way endoscope steering mechanism, comprising:
a plurality of hollow steering members, each having a plurality of hollow positioning portions and a plurality of connecting portions, and the connecting portions including a first connecting portion, a second connecting portion, a third connecting portion and a fourth connecting portion, and the first connecting portion and the second connecting portion being arranged opposite to each other and disposed on a side of the hollow steering member, and the third connecting portion and the fourth connecting portion being arranged opposite to each other and disposed on the other side of the hollow steering member, and the first connecting portion and the second connecting portion being arranged opposite to the third connecting portion and the fourth connecting portion respectively;
a plurality of two-way flexible plastic members, each including a first two-way flexible plastic member and a second two-way flexible plastic member, and the two-way flexible plastic member being partially bent, and the bending and steering of the plurality of two-way flexible plastic members being accumulated to define a partial bending of the two-way flexible plastic member, and an end of the first two-way flexible plastic member being coupled to the first connecting portion, and the other end of the first two-way flexible plastic member being coupled to the third connecting portion adjacent to another hollow steering member, and an end of the second two-way flexible plastic member being coupled to the second connecting portion, and the other end of the second two-way flexible plastic member being coupled to the fourth connecting portion adjacent to another hollow steering member;
a plurality of guiding wire members, passed and installed into the plurality of corresponding hollow positioning portions respectively; and
a plurality of densely wound coils, coupled to an end of a steering module, and the steering module being formed by arranging and coupling the plurality of two-way flexible plastic members to the plurality of hollow steering members, and the densely wound coil being extended in a direction opposite to the steering module from an end of the steering module and sheathed on the corresponding guiding wire member;
thereby, when one of the guiding wire members is pulled, the first two-way flexible plastic member and the second two-way flexible plastic member are pulled to bend in a first direction or a second direction to turn the steering module.

2. The two-way endoscope steering mechanism of claim 1, wherein the first two-way flexible plastic member has an end integrally formed with the first connecting portion and the third connecting portion, and the second two-way flexible plastic member has an end integrally formed with the second connecting portion and the fourth connecting portion.

3. The two-way endoscope steering mechanism of claim 1, wherein the hollow positioning portion is a through hole.

4. The two-way endoscope steering mechanism of claim 1, wherein the hollow positioning portion is an open slot, and each open slot has an opening arranged with a rotation angle with respect to an opening of another adjacent open slot, such that the guiding wire member is positioned in the hollow positioning portion.

5. A four-way endoscope steering mechanism, comprising:
a plurality of hollow steering members, each having a plurality of hollow positioning portions and a plurality of connecting portions, and each connecting portion including a first connecting portion, a second connecting portion, a third connecting portion and a fourth connecting portion, and the first connecting portion and the second connecting portion being arranged opposite to each other and disposed on a side of the hollow steering member, and the third connecting portion and the fourth connecting portion being arranged opposite to each other and disposed on the other side of the hollow steering member, and the third connecting portion forming an angle with respect to the first connecting portion, and the fourth connecting portion forming another angle with respect to the second connecting portion;
a plurality of two-way flexible plastic members, each having a first two-way flexible plastic member and a second two-way flexible plastic member, and the two-way flexible plastic member being partially bent, and an end of the first two-way flexible plastic member being coupled to the first connecting portion, and the other end of the first two-way flexible plastic member being coupled to the third connecting portion adjacent to the other hollow steering member, and an end of the second two-way flexible plastic member being coupled to the second connecting portion, and the other end of the second two-way flexible plastic member being coupled to the fourth connecting portion adjacent to another hollow steering member, so that the two-way flexible plastic members on a side of the hollow steering member are perpendicular to the two-way flexible plastic members on the other side of the hollow steering member;
a plurality of guiding wire members, passed and installed in the plurality of corresponding hollow positioning portions; and
a plurality of densely wound coils, coupled to an end of a steering module, and the steering module being formed by arranging and coupling the plurality of two-way flexible plastic members to the plurality of hollow steering members respectively, and the densely wound coil being extended in an opposite direction of the steering module from an end of the steering module and sheathed on the corresponding guiding wire member;
thereby, when one of the guiding wire members is pulled, the guiding wire member drives the plurality of corresponding hollow steering members to bend the plurality of two-way flexible plastic members on a side of the hollow steering member in a first direction or a second direction, or drives the plurality of two-way flexible plastic members on the other side of the hollow steering member to bend in a third direction or a fourth direction to turn the steering module.

6. The four-way endoscope steering mechanism of claim 5, wherein the first two-way flexible plastic member has an end integrally formed with the first connecting portion and the third connecting portion, and the second two-way flexible plastic member has an end integrally formed with the second connecting portion and the fourth connecting portion.

7. The four-way endoscope steering mechanism of claim 5, wherein the hollow positioning portion is a through hole.

8. The four-way endoscope steering mechanism of claim 5, wherein the hollow positioning portion is an open slot, and each open slot has an opening arranged with a rotation angle with respect to an opening of another adjacent open slot, such that the guiding wire member is positioned in the hollow positioning portion.
